(19) United States
(12) Patent Application Publication
(10) Pub. No.: US 2002/0032147 A1
(43) Pub. Date: Mar. 14, 2002

(54) Perfume composition and cleaning compositions comprising the perfume composition

(75) Inventors: Peter Robert Foley, Cincinnati, OH (US); Carl-Eric Kaiser, Mason, OH (US); Zaiyou Liu, West Chester, OH (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(73) Assignee: The procter & Gamble Company

(21) Appl. No.: 09/904,227

(22) Filed: Jul. 12, 2001

(30) Foreign Application Priority Data
Jul. 13, 2000 (US) PCT/US00/19078

(51) Int. Cl. A61K 7/46

(52) U.S. Cl. 512/25; 512/27

(57) ABSTRACT

The present invention relates to perfume composition and a cleaning composition comprising the perfume composition. The perfume composition comprises at least 7.5% by weight of the composition of a first perfume ingredient having boiling point of 250° C. or less and ClogP of 3.0 or less, and at least 35% by weight of the composition of a second perfume ingredient having boiling point of 250° C. or less and ClogP of 3.0 or more. The composition also comprises at least one first or second perfume ingredient is present in an amount of at least 7% by weight of the composition.

### TECHNICAL FIELD

The present application relates to a perfume composition comprising “blooming” perfume ingredients to provide a high intensity and long-lasting perfume aroma. The present application also relates to a cleaning composition comprising such a perfume composition and the use of the cleaning composition in hand-dishwashing.

### BACKGROUND

In many parts of the world, liquid detergent products are used for the hand washing of eating, dishware and cooking utensils. Since dishwashing operations using such products involve the close proximity of the dishwasher to the dishwashing operation and to the detergent products used therein, it is desirable and commercially beneficial to add perfume materials to such products. Perfumes provide an olfactory aesthetic benefit that can not only provide the user with a pleasant aroma, but also serve as a signal of cleanliness. Such perfumes should therefore be readily noticeable to the user of the dishwashing products. Any perfumes that are used, however, should not leave residue or residual odor on surfaces that the washing solution formed from any such product has contacted. Residual perfumes on such surfaces, for example, on pots, pans, dishes and countertops, may be construed negatively by consumers as chemical residues, and may result in concerns around food contamination in subsequent uses.

Given the foregoing considerations, it is an object of the present invention to provide a perfume composition which not only provides the user with a readily noticeable, pleasant and desirable odor during use, but which also impart a readily noticeable and desirable odor to the surrounding area during and after use. It is thus also an object of the present invention to provide a perfume which is capable of masking malodors generated during the cooking, eating and cleaning process.

It is a further object of the present invention to provide such blooming perfumed detergent products that do not leave undesirable residual odor on surfaces contacted with washing solutions formed from such products.

### SUMMARY OF THE INVENTION

According to the present invention there is provided a perfume composition comprising:

a) at least 7.5% by weight of the composition of a first perfume ingredient having boiling point of 250° C. or less and ClogP of 3.0 or less, and;

b) at least 35% by weight of the composition of a second perfume ingredient having boiling point of 250° C. or less and ClogP of 3.0 or more, the composition being further characterised in that at least one first or second perfume ingredient is present in an amount of at least 7% by weight of the composition.

### DETAILED DESCRIPTION OF THE INVENTION

A blooming perfume composition is one which comprises blooming perfume ingredients. A blooming perfume ingredient may be characterized by its boiling point (B.P.) and its octanol/water partition coefficient (P). B.P. according to the present invention is measured under normal standard pressure of 760 mmHg. The boiling points of many perfume ingredients, at standard 760 mmHg are given in, e.g., “Perfume and Flavor Chemicals (Aroma Chemicals),” Steffen Arctander, published by the author, 1969, incorporated herein by reference.

The octanol/water partition coefficient of a perfume ingredient is the ratio between its equilibrium concentrations in octanol and in water. The partition coefficients of the preferred perfume ingredients of the present invention may be more conveniently given in the form of their logarithm to the base 10, logP. The logP values of many perfume ingredients have been reported; for example, the Pomona92 database, available from Daylight Chemical Information Systems, Inc. (Daylight CIS), Irvine, Calif., contains many, along with citations to the original literature. However, the logP values are most conveniently calculated by the “CLOGP” program, also available from Daylight CIS. This program also lists experimental logP values when they are available in the Pomona database. The “calculated logP” (ClogP) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990, incorporated herein by reference). The fragment approach is based on the chemical structure of each perfume ingredient, and takes into account the numbers and types of atoms, the atom connectivity, and chemical bonding. The ClogP values, which are the most reliable and widely used estimates for this physicochemical property, are preferably used instead of the experimental logP values in the selection of perfume ingredients which are useful in the present invention.

According to a first aspect of the present invention the perfume composition comprising at least two perfume ingredients. The first perfume ingredient is characterised by having boiling point of 250° C. or less and ClogP of 3.0 or less. More preferably the first perfume ingredient has boiling point of 240° C. or less, most preferably 235° C. or less. More preferably the first perfume ingredient has a ClogP value of less than 3.0, more preferably 2.5 or less. The first perfume ingredient is present at a level of at least 7.5% by weight of the composition, more preferably at least 8.5% and most preferably at least 9.5% by weight of the composition.

The second perfume ingredient is characterised by having boiling point of 250° C. or less and ClogP of 3.0 or more. More preferably the second perfume ingredient has boiling point of 240° C. or less, most preferably 235° C. or less. More preferably the second perfume ingredient has a ClogP value of greater than 3.0, even more preferably greater than 3.2. The second perfume ingredient is present at a level of at least 35% by weight of the composition, more preferably at least 37.5% and most preferably greater than 40% by weight of the perfume composition.

The perfume composition of the present invention comprises at least one perfume from the first group of perfume ingredients and at least one perfume from the second group of perfume ingredients. More preferably the perfume composition comprises a plurality of ingredients chosen from the first group of perfume ingredients and a plurality of ingredients chosen from the second group of perfume ingredients.

In addition to the above, it is also required that the composition comprise at least one perfume ingredient selected from either first and/or second perfume ingredients which is present in an amount of at least 7% by weight of the perfume composition, preferably at least 8.5% of the perfume composition, and most preferably, at least 10% of the perfume composition.

The compositions of the present invention differ from those blooming perfume compositions previously described in that unusually high levels of particularly chosen perfume ingredients are used. Such high levels of perfume had not previously been used because of a phenomenon known as the odor detection threshold. Perfume raw material generates an olfactory response in the individual smelling the perfume. The minimum concentration of perfume ingredient which is consistently perceived to generate an olfactory response in an individual, is known as the Odor Detection threshold (ODT). As the concentration of perfume is increased so is the odor intensity of the perfume, and the olfactory response of the individual. This is so until the concentration of the perfume reaches a maximum, at which point the odor intensity reaches a plateau beyond which there is no additional olfactory response by the individual. This range of perfume concentration through which the individual consistently perceives an odor is known as the Odor Detection Range (ODR).

It had been understood, until now, that the concentration of perfume ingredients in the perfume composition should be formulated within the ODR of the perfume ingredient, since any composition comprising higher levels provide no additional olfactory response and are thus costly and inefficient.

The Applicants have however found that it is in fact beneficial to exceed the ODR of the perfume ingredient. The perfume is not only effusive and very noticeable when the product is used, but it has also been found that the perfume forms a reservoir in the headspace. By ‘headspace’ it is meant the immediate atmosphere surrounding the perfume, hence the headspace may be the atmosphere in the container in which the composition is stored or the atmosphere between the washing-up basin and the user. The reservoir of perfume serves to replace diffused perfume, thus maintaining perfume concentration in the headspace at or beyond the odor detection threshold of the perfume throughout use, and preferably, after use. Moreover, is has also been found that the perfume tends to linger for longer in the room in which the composition is used. Thus in a preferred embodiment at least one perfume ingredient selected from the first and/or second perfume ingredients is preferably present at a level of 50% in excess of the ODR, more preferably 150% in excess of the ODR. For very lingering perfume, at least one perfume ingredient should be added at a level of more than 300% of the ODR.

Odor detection thresholds are determined using a gas chromatograph. The gas chromatograph is calibrated to determine the exact volume of material injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of material. To determine whether a material has a threshold below 50 ppb, solutions are delivered to the sniff port at the back-calculated concentration. A panelist sniffs the GC effluent and identifies the retention time when odor is noticed. The average across all panelists determines the threshold of noticeability.

The necessary amount of analyte is injected onto the column to achieve a 50 ppb concentration at the detector. Typical gas chromatograph parameters for determining odor detection thresholds are listed below.

GC: 5890 Series II with FID detector

7673 Autosampler

Column: J&W Scientific DB-1

Length 30 meters ID 0.25 mm film thickness 1 micron

Method

Split Injection: 17/1 split ratio

Autosampler: 1.13 microliters per injection

Column Flow: 1.10 mL/minute

Air Flow: 345 minute

Inlet Temp. 245° C.

Detector Temp. 285° C.

Temperature Information

Initial Temperature: 50° C.

Rate: 5 C./minute

Final Temperature: 280° C.

Final Time: 6 minutes

Leading assumptions:

(i) 12 seconds per sniff

(ii) GC air adds to sample dilution

The first and second perfume ingredients of the present invention are preferably selected from the group consisting of esters, ketones, aldehydes, alcohols, derivatives thereof and mixtures thereof. Table 1 provides some examples of preferred first perfume ingredients and table 2 provides some examples of preferred second perfume ingredients.

In a preferred embodiment the preferred weight ratio of second perfume ingredients to first blooming perfume ingredient is typically at least 1, preferably at least 1.3, more preferably 1.5, and even more preferably 2. The perfume compositions comprises at least 42.5%, more preferably at least 50%, even more preferably at least 60% of the combined first and second perfume ingredients.

TABLE 1
Examples of First Perfume Ingredients

| | Approx | Approx |
|---|---|---|
| Perfume Ingredients | BP (° C.) | ClogP |
| Allyl Caproate | 185 | 2.772 |
| Amyl Acetate | 142 | 2.258 |
| Amyl Propionate | 161 | 2.657 |
| Anisic Aldehyde | 248 | 1.779 |
| Anisole | 154 | 2.061 |
| Benzaldehyde | 179 | 1.480 |
| Benzyl Acetate | 215 | 1.960 |
| Benzyl Acetone | 235 | 1.739 |
| Benzyl Alcohol | 205 | 1.100 |
| Benzyl Formate | 202 | 1.414 |
| Benzyl Iso Valerate | 246 | 2.887 |
| Benzyl Propionate | 222 | 2.489 |
| Beta Gamma Hexenol | 157 | 1.337 |
| Camphor Gum | 208 | 2.117 |
| laevo-Carveol | 227 | 2.265 |
| d-Carvone | 231 | 2.010 |
| laevo-Carvone | 230 | 2.203 |
| Cinnamyl Formate | 250 | 1.908 |
| cis-Jasmone | 248 | 2.712 |
| cis-3-Hexenyl Acetate | 169 | 2.243 |
| Cuminic alcohol | 248 | 2.531 |
| Cuminic aldehyde | 236 | 2.780 |
| Cyclal C | 180 | 2.301 |
| Dimethyl Benzyl Carbinol | 215 | 1.891 |
| Dimethyl Benzyl Carbinyl Acetate | 250 | 2.797 |
| Ethyl Acetate | 77 | 0.730 |
| Ethyl Aceto Acetate | 181 | 0.333 |
| Ethyl Amyl Ketone | 167 | 2.307 |
| Ethyl Benzoate | 212 | 2.640 |
| Ethyl Butyrate | 121 | 1.729 |
| Ethyl Hexyl Ketone | 190 | 2.916 |
| Ethyl-2-methyl butyrate | 131 | 2.100 |
| Ethyl Methyl Pentanoate | 143 | 2.700 |
| Ethyl Phenyl Acetate | 229 | 2.489 |
| Eucalyptol | 176 | 2.756 |
| Fenchyl Alcohol | 200 | 2.579 |
| Flor Acetate (tricyclo Decenyl Acetate) | 175 | 2.357 |
| Frutene (tricyclo Decenyl Propionate) | 200 | 2.260 |
| Geraniol | 230 | 2.649 |
| Hexenol | 159 | 1.397 |
| Hexenyl Acetate | 168 | 2.343 |
| Hexyl Acetate | 172 | 2.787 |
| Hexyl Formate | 155 | 2.381 |
| Hydratropic Alcohol | 219 | 1.582 |
| Hydroxycitronellal | 241 | 1.541 |
| Isoamyl Alcohol | 132 | 1.222 |
| Isomenthone | 210 | 2.831 |
| Isopulegyl Acetate | 239 | 2.100 |
| Isoquinoline | 243 | 2.080 |
| Ligustral | 177 | 2.301 |
| Linalool | 198 | 2.429 |
| Linalool Oxide | 188 | 1.575 |
| Linalyl Formate | 202 | 2.929 |
| Menthone | 207 | 2.650 |
| Methyl Acetophenone | 228 | 2.080 |
| Methyl Amyl Ketone | 152 | 1.848 |
| Methyl Anthranilate | 237 | 2.024 |
| Methyl Benzoate | 200 | 2.111 |
| Methyl Benzyl Acetate | 213 | 2.300 |
| Methyl Eugenol | 249 | 2.783 |
| Methyl Heptenone | 174 | 1.703 |
| Methyl Heptine Carbonate | 217 | 2.528 |
| Methyl Heptyl Ketone | 194 | 1.823 |
| Methyl Hexyl Ketone | 173 | 2.377 |
| Methyl Phenyl Carbinyl Acetate | 214 | 2.269 |
| Methyl Salicylate | 223 | 1.960 |
| Nerol | 227 | 2.649 |
| Octalactone | 230 | 2.203 |
| Octyl Alcohol (Octanol-2) | 179 | 2.719 |
| para-Cresol | 202 | 1.000 |
| para-Cresyl Methyl Ether | 176 | 2.560 |
| para-Methyl Acetophenone | 228 | 2.080 |
| Phenoxy Ethanol | 245 | 1.188 |
| Phenyl Acetaldehyde | 195 | 1.780 |
| Phenyl Ethyl Acetate | 232 | 2.129 |
| Phenyl Ethyl Alcohol | 220 | 1.183 |
| Phenyl Ethyl Dimethyl Carbinol | 238 | 2.420 |
| Prenyl Acetate | 155 | 1.684 |
| Propyl Butyrate | 143 | 2.210 |
| Pulegone | 224 | 2.350 |
| Rose Oxide | 182 | 2.896 |
| Safrole | 234 | 1.870 |
| 4-Terpinenol | 212 | 2.749 |
| alpha-Terpineol | 219 | 2.569 |
| Viridine | 221 | 1.293 |

TABLE 2
Examples of Second Perfume Ingredients

| | Approx. | Approx. |
|---|---|---|
| Perfume Ingredients | BP (° C.) | ClogP |
| allo-Ocimene | 192 | 4.362 |
| Allyl Heptoate | 210 | 3.301 |
| Anethol | 236 | 3.314 |
| Benzyl Butyrate | 240 | 3.698 |
| Camphene | 159 | 4.192 |
| Carvacrol | 238 | 3.401 |
| cis-3-Hexenyl Tiglate | 101 | 3.700 |
| Citral (Neral) | 228 | 3.120 |
| Citronellol | 225 | 3.193 |
| Citronellyl Acetate | 229 | 3.670 |
| Citronellyl Isobutyrate | 249 | 4.937 |
| Citronellyl Nitrile | 225 | 3.094 |
| Citronellyl Propionate | 242 | 4.628 |
| Cyclohexyl Ethyl Acetate | 187 | 3.321 |
| Decyl Aldehyde | 209 | 4.008 |
| Delta Damascone | 242 | 3.600 |
| Dihydro Myrcenol | 208 | 3.030 |
| Dihydromyrcenyl Acetate | 225 | 3.879 |
| Dimethyl Octanol | 213 | 3.737 |
| Fenchyl Acetate | 220 | 3.485 |
| gamma Methyl Ionone | 230 | 4.089 |
| gamma-Nonalactone | 243 | 3.140 |
| Geranyl Acetate | 245 | 3.715 |
| Geranyl Formate | 216 | 3.269 |
| Geranyl Isobutyrate | 245 | 4.393 |
| Geranyl Nitrile | 222 | 3.139 |
| Hexenyl Isobutyrate | 182 | 3.181 |
| Hexyl Neopentanoate | 224 | 4.374 |
| Hexyl Tiglate | 231 | 3.800 |
| alpha-Ionone | 237 | 3.381 |
| beta-Ionone | 239 | 3.960 |
| gamma-Ionone | 240 | 3.780 |
| alpha-Irone | 250 | 3.820 |
| Isobornyl Acetate | 227 | 3.485 |
| Isobutyl Benzoate | 242 | 3.028 |
| Isononyl Acetate | 200 | 3.984 |
| Isononyl Alcohol | 194 | 3.078 |
| Isomenthol | 219 | 3.030 |
| para-Isopropyl Phenylacetaldehyde | 243 | 3.211 |
| Isopulegol | 212 | 3.330 |
| Lauric Aldehyde (Dodecanal) | 249 | 5.066 |
| d-Limonene | 177 | 4.232 |
| Linalyl Acetate | 220 | 3.500 |
| Menthyl Acetate | 227 | 3.210 |
| Methyl Chavicol | 216 | 3.074 |
| alpha-iso “gamma” Methyl Ionone | 230 | 4.209 |
| Methyl Nonyl Acetaldehyde | 232 | 4.846 |
| Methyl Octyl Acetaldehyde | 228 | 4.317 |
| Myrcene | 167 | 4.272 |
| Neral | 228 | 3.120 |
| Neryl Acetate | 231 | 3.555 |
| Nonyl Acetate | 212 | 4.374 |
| Nonyl Aldehyde | 212 | 3.479 |
| Octyl Aldehyde | 223 | 3.845 |
| Orange Terpenes (d-Limonene) | 177 | 4.232 |
| para-Cymene | 179 | 4.068 |
| Phenyl Ethyl Isobutyrate | 250 | 3.000 |
| alpha-Pinene | 157 | 4.122 |
| beta-Pinene | 166 | 4.182 |
| alpha-Terpinene | 176 | 4.412 |
| gamma-Terpinene | 183 | 4.232 |
| Terpinolene | 184 | 4.232 |
| Terpinyl acetate | 220 | 3.475 |
| Tetrahydro Linalool | 191 | 3.517 |
| Tetrahydro Myrcenol | 208 | 3.517 |
| Undecenal | 223 | 4.053 |
| Veratrol | 206 | 3.140 |
| Verdox | 221 | 4.059 |
| Vertenex | 232 | 4.060 |

TABLE 3

| | Approximate | Approx. |
|---|---|---|
| Perfume Ingredients | B.P. (° C.) | ClogP |
| Allyl Cyclohexane Propionate | 267 | 3.935 |
| Ambrettolide | 300 | 6.261 |
| Amyl Benzoate | 262 | 3.417 |
| Amyl Cinnamate | 310 | 3.771 |
| Amyl Cinnamic Aldehyde | 285 | 4.324 |
| Amyl Cinnamic Aldehyde Dimethyl Acetal | 300 | 4.033 |
| iso-Amyl Salicylate | 277 | 4.601 |
| Aurantiol | 450 | 4.216 |
| Benzophenone | 306 | 3.120 |
| Benzyl Salicylate | 300 | 4.383 |
| Cadinene | 275 | 7.346 |
| Cedrol | 291 | 4.530 |
| Cedryl Acetate | 303 | 5.436 |
| Cinnamyl Cinnamate | 370 | 5.480 |
| Coumarin | 291 | 1.412 |
| Cyclohexyl Salicylate | 304 | 5.265 |
| Cyclamen Aldehyde | 270 | 3.680 |
| Dihydro Isojasmonate | +300 | 3.009 |
| Diphenyl Methane | 262 | 4.059 |
| Ethylene Brassylate | 332 | 4.554 |
| Ethyl Methyl Phenyl Glycidate | 260 | 3.165 |
| Ethyl Undecylenate | 264 | 4.888 |
| iso-Eugenol | 266 | 2.547 |
| Exaltolide | 280 | 5.346 |
| Galaxolide | +260 | 5.482 |
| Geranyl Anthranilate | 312 | 4.216 |
| Hexadecanolide | 294 | 6.805 |
| Hexenyl Salicylate | 271 | 4.716 |
| Hexyl Cinnamic Aldehyde | 305 | 5.473 |
| Hexyl Salicylate | 290 | 5.260 |
| Linalyl Benzoate | 263 | 5.233 |
| 2-Methoxy Naphthalene | 275 | 3.235 |
| Methyl Cinnamate | 263 | 2.620 |
| Methyl Dihydrojasmonate | +300 | 2.275 |
| beta-Methyl Naphthyl ketone | 300 | 2.275 |
| Musk Indanone | +250 | 5.458 |
| Musk Ketone | MP = 137° C. | 3.014 |
| Musk Tibetine | MP = 136° C. | 3.831 |
| Myristicin | 276 | 3.200 |
| delta-Nonalactone | 280 | 2.760 |
| Oxahexadecanolide-10 | +300 | 4.336 |
| Oxahexadecanolide-11 | MP = 35° C. | 4.336 |
| Patchouli Alcohol | 285 | 4.530 |
| Phantolide | 288 | 5.977 |
| Phenyl Ethyl Benzoate | 300 | 4.058 |
| Phenylethylphenylacetate | 325 | 3.767 |
| alpha-Santalol | 301 | 3.800 |
| Thibetolide | 280 | 6.246 |
| delta-Undecalactone | 290 | 3.830 |
| gamma-Undecalactone | 297 | 4.140 |
| Vanillin | 285 | 1.580 |
| Vetiveryl Acetate | 285 | 4.882 |
| Yara-Yara | 274 | 3.235 |

(a) M.P. is melting point; these ingredients have a B.P. higher than 275° C.

### Examples of Optional Perfume Ingredients

In the perfume art, some auxiliary materials having no odor, or a low odor, are used, e.g., as solvents, diluents, extenders or fixatives. Non-limiting examples of these materials are ethyl alcohol, carbitol, diethylene glycol, dipropylene glycol, diethyl phthalate, triethyl citrate, isopropyl myristate, and benzyl benzoate. These materials are used for, e.g., solubilizing or diluting some solid or viscous perfume ingredients to, e.g., improve handling and/or formulating. These materials are useful in the blooming perfume compositions, but are not counted in the calculation of the limits for the definition/formulation of the blooming perfume compositions of the present invention.

Optional, including non-blooming perfume ingredients, which should be minimized in hand dish particulate cleaning compositions of the present invention, are those having a B.P. of more than 275° C. Table 3 gives some non-limiting examples of optional perfume ingredients.

It can be desirable to use blooming and delayed blooming perfume ingredients and even other ingredients, preferably in small amounts, in the blooming perfume compositions of the present invention, that have low odor detection threshold values. The odor detection threshold of an odorous material is the lowest vapor concentration of that material which can be detected. The odor detection threshold and some odor detection threshold values are discussed in, e.g., “Standardized Human Olfactory Thresholds”, M. Devos et al, IRL Press at Oxford University Press, 1990, and “Compilation of Odor and Taste Threshold Values Data”, F. A. Fazzalari, editor, ASTM Data Series DS 48A, American Society for Testing and Materials, 1978, both of said publications being incorporated by reference. The use of small amounts of non-blooming perfume ingredients that have low odor detection threshold values can improve perfume odor character, without the potential negatives normally associated with such ingredients, e.g., spotting and/or filming on, e.g., dish surfaces. Non-limiting examples of perfume ingredients that have low odor detection threshold values useful in the present invention include coumarin, vanillin, ethyl vanillin, methyl dihydro isojasmonate, 3-hexenyl salicylate, isoeugenol, lyral, gamma-undecalactone, gamma-dodecalactone, methyl beta naphthyl ketone, and mixtures thereof. These materials are preferably present at low levels in addition to the blooming and optionally delayed blooming ingredients, typically less than 5%, preferably less than 3%, more preferably less than 2%, by weight of the blooming perfume compositions of the present invention.

The perfumes suitable for use in the cleaning compositions herein can be formulated from known fragrance ingredients and for purposes of enhancing environmental compatibility, the perfume compositions used herein are preferably substantially free of halogenated fragrance materials and nitromusks.

Alternatively the perfume ingredients of the present invention or a portion thereof may be complexed with a complexing agent. Complexing agents may include any compound which encapsulate or bind perfume raw materials in aqueous solution. Binding can result from one or more of strong reversible chemical bonding, reversible weak chemical bonding, weak or strong physical absorption or adsorption and, for example, may take the form of encapsulation, partial encapsulation, or binding. Complexes formed can be 1:1, 1:2, 2:1 complexant:perfume ratios, or can be more complex combinations. It is also possible to bind perfumes via physical encapsulation via coating (e.g. starch coating), or coacervation. Key to effective complexation for controlled perfume release is an effective de-complexation mechanism, driven by use of the product for washing dishes or hard surfaces. Suitable de-complexation mechanisms can include dilution in water, increased or decreased temperature, increased or decreased ionic strength. It is also possible to chemically or physically decompose a coated perfume, eg via reaction with enzyme, bleach or alkalinity, or via solubilization by surfactants or solvents. Preferred complexing agents include cyclodextrin, zeolites, coacervates starch coatings, and mixtures thereof. Cyclodextrin molecules are known for their ability to form complexes with perfume ingredients and have typically been taught as a perfume carrier. In addition, cyclodextrin molecules also appear to be surprisingly effective at reducing malodors generated by nitrogenous compounds, such as amines.

The prior art teaches the use of drier-added fabric softener sheets containing high levels of cyclodextrin/perfume complexes wherein the fabrics treated with this solid cyclodextrin complex release perfume when the fabrics are rewetted and that these cyclodextrin/perfume complexes can be used in aqueous rinse-added fabric softener compositions without being protected. By “protected” it is meant that the cyclodextrin is encapsulated in a hydrophobic wax coating so the cyclodextrin/perfume complexes will not decompose due to the presence of water. See e.g. U.S. Pat. No. 5,578,563, issued Nov. 26, 1996, to Trinh et al., which is hereby incorporated by reference. Thus the cyclodextrin used in the present invention may either be “unprotected”, as discussed above, or “protected” by the hydrophobic-coating protection techniques discussed in the prior art; see e.g. U.S. Pat. No. 5,102,564 to Gardlik et al., issued Apr. 7, 1992; U.S. Pat. No. 5,234,610, to Gardlik et al., issued Aug. 10, 1993.

The cavity of a cyclodextrin molecule has a substantially conical shape. It is preferable in the present invention that the cone-shaped cavity of the cyclodextrins have a length (altitude) of 8 Å and a base size of from 5 Å to 8.5 Å. Thus the preferred cavity volume for cyclodextrins of the present invention is from 65 Å³ to 210 Å³.

Suitable cyclodextrin species include any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

The cyclodextrin molecules are preferably water-soluble. The water-soluble cyclodextrins used herein preferably have a water solubility of at least 10 g in 100 ml water, more preferably at least 25 g in 100 ml of water at standard temperature and pressure. Examples of preferred water-soluble cyclodextrin derivative species suitable for use herein are hydroxypropyl alpha-cyclodextrin, methylareal alpha-cyclodextrin, methylated beta-cyclodextrin, hydroxyethyl beta-cyclodextrin, and hydroxypropyl beta-cyclodextrin. Hydroxyalkyl cyclodextrin derivatives preferably have a degree of substitution of from 1 to 14, more preferably from 1.5 to 7, wherein the total number of OR groups per cyclodextrin is defined as the degree of substitution. Methylated cyclodextrin derivatives typically have a degree of substitution of from 1 to 18, preferably from 3 to 16. A known methylated beta-cyclodextrin is heptakis-2,6-di-O-methyl-.beta.-cyclodextrin, commonly known as DIMEB, in which each glucose unit has 2 methyl groups with a degree of substitution of 14. A preferred, more commercially available methylated beta-cyclodextrin is a randomly methylated beta-cyclodextrin having a degree of substitution of 12.6. The preferred cyclodextrins are available, e.g., from American Maize-Products Company and Wacker Chemicals (USA), Inc.

Further cyclodexrin species suitable for use in the present invention include alpha-cyclodextrin and derivatives thereof, gamma-cyclodextrin and derivatives thereof, derivatised beta-cyclodextrins, and/or mixtures thereof. Other derivatives of cyclodextrin which are suitable for use in the present invention are discussed in U.S. Pat. No. 5,578,563, incorporated above. It should be noted that two or more different species of cyclodextrin may be used in the same liquid detergent composition.

The complexes may be formed in any of the ways known in the art. Typically, the complexes are formed either by bringing the fragrance materials and the cyclodextrin together in a suitable solvent e.g. water and ethanol mixtures, propylene glycol. Additional examples of suitable processes as well as further preferred processing parameters and conditions are disclosed in U.S. Pat. No. 5,234,610, to Gardlik et al., issued Aug. 10, 1993, which is hereby incorporated by reference. After the cyclodextrin and fragrance materials are mixed together, this mixture is added to the liquid detergent composition.

Generally, only a portion (not all) of the fragrance materials mixed with the cyclodextrin will be encapsulated by the cyclodextrin and form part of the cyclodextrin/perfume complex; the remaining fragrance materials will be free of the cyclodextrin and when the cyclodextrin/perfume mixture is added to the detergent composition they will enter the detergent composition as free perfume molecules. A portion of free cyclodextrin molecules which are not complexed with the fragrance materials may also be present. In an alternative embodiment of the present invention, the fragrance materials and cyclodextrins are added uncomplexed and separately to the liquid detergent compositions. Consequently, the cyclodextirns and fragrance materials will come into the presence of each other in the composition, and a portion of each will combine to form the desired fragrance materials/cyclodextrin complex.

Suitable fragrance materials for use in the present invention are described in greater detail below.

In general, perfume/cyclodextrin complexes have a molar ratio of perfume compound to cyclodextrin of 1:1. However, the molar ratio can be either higher or lower, depending on the size of the perfume compound and the identity of the cyclodextrin compound. In the present invention the molar ratio of fragrance materials to cyclodextrin is preferably from 4:1 to 1:4, more preferably from 1.5:1 to 1:2, most preferably from 1:1 to 1:1.5. The molar ratio can be determined easily by forming a saturated solution of the cyclodextrin and adding the perfume to form the complex. In general the complex will precipitate readily. If not, the complex can usually be precipitated by the addition of electrolyte, change of pH, cooling, etc. The complex can then be analyzed to determine the ratio of perfume to cyclodextrin.

The actual complexes are determined by the size of the cavity in the cyclodextrin and the size of the perfume molecule. Although the normal complex is one molecule of perfume in one molecule of cyclodextrin, complexes can be formed between one molecule of perfume and two molecules of cyclodextrin when the perfume molecule is large and contains two portions that can fit in the cyclodextrin. Highly desirable complexes can be formed using mixtures of cyclodextrins since perfumes are normally mixtures of materials that vary widely in size. It is usually desirable that at least a majority of the material be beta- and/or gamma-cyclodextrin. It is highly desirable to use the reaction mixtures from the intermediate stages of the manufacture of the pure cyclodextrins as discussed hereinbefore.

### EXAMPLES

The following examples numbered A to H, of the preferred perfume composition are in no way meant to be limiting.

| Perfume | | | | | | | | |
|---|---|---|---|---|---|---|---|---|
| ingredient | A | B | C | D | E | F | G | H |
| Allyl Caproate | 2 | — | — | 4 | — | 2 | — | 3 |
| Citronellyl | 5 | 8 | 6 | 3 | 5 | 6 | 5 | 3 |
| Acetate | | | | | | | | |
| Delta | 1 | 0.5 | 0.9 | 3 | 0.8 | 2 | 0.6 | 1 |
| Damascone | | | | | | | | |
| Ethyl-2-methyl | 8 | 2 | 1.5 | 12 | 1.5 | 15 | 1 | 11 |
| Butyrate | | | | | | | | |
| Flor Acetate | 8 | — | — | 4 | — | 4 | — | 5 |
| Frutene | 4 | — | — | 8 | — | 4 | — | 8 |
| Geranyl Nitrile | 1 | 15 | 22 | 1 | 28 | 1 | 32 | 5 |
| Ligustral | 6 | 7.5 | 12 | 10 | 8 | 13 | 8 | 10 |
| Methyl dihydro | 27.69 | 37.36 | 21.89 | 25 | 28.04 | 30 | 25.70 | 25.59 |
| Jasmonate | | | | | | | | |
| Nectaryl | 5 | — | — | 3 | — | 4 | — | 3 |
| Neobutanone | 0.30 | 0.09 | 0.12 | 0.3 | 0.1 | 0.2 | 0.15 | 0.4 |
| Oxane | 0.01 | 0.05 | 0.09 | 0.01 | 0.06 | 0.01 | 0.05 | 0.01 |
| Tetrahydro | 32 | — | — | 26.69 | — | 18.79 | — | 25 |
| Linalool | | | | | | | | |
| Methyl nonyl | — | 7 | 15 | — | 10 | — | 8.5 | — |
| acetaldehyde | | | | | | | | |
| Ethyl-2-methyl | — | 1 | 1.5 | — | 1 | — | 1 | — |
| pentanoate | | | | | | | | |
| Iso E Super | — | 3 | 2 | — | 3 | — | 3 | — |
| Ionone beta | — | 1.5 | 2 | — | 1.5 | — | 1 | — |
| Habanolide | — | 3 | 3 | — | 3 | — | 3 | — |
| Geraniol | — | 15 | 12 | — | 10 | — | 11 | — |

Cleaning Composition

In a second aspect, the present invention relates to a cleaning composition comprising the perfume composition described above. The cleaning composition is preferably suitable for use in cleaning hard surfaces, for example any kind of surfaces typically found in houses like kitchens, bathrooms, or in car interiors or exteriors, e.g., floors, walls, tiles, windows, sinks, showers, shower plastified curtains, wash basins, WCs, dishes, fixtures and fittings and the like made of different materials like ceramic, vinyl, no-wax vinyl, linoleum, melamine, glass, any plastics, plastified wood, metal or any painted or varnished or sealed surface and the like. Hard-surfaces also include household appliances including, but not limited to, refrigerators, freezers, washing machines, automatic dryers, ovens, microwave ovens, dishwashers and so on. More preferably the cleaning composition according to the present invention is suitable for cleaning dishware including dishes, cups, cutlery, glasses, food storage containers, cutlery, cooking utensils, sinks and other kitchen surfaces.

The cleaning composition, may be in any suitable form for example, particulate, gel, paste or liquid. The cleaning composition is preferably in liquid form. Moreover the cleaning composition is preferably in liquid aqueous form. Where present water is preferably present at a level of from 30 to 80% by weight of the cleaning composition, more preferably from 40 to 70% and most preferably from 45 to 65%.

The cleaning composition comprises as an essential features thereof from 0.005 to 2% of perfume composition as described herein. More preferably, the cleaning composition comprises from 0.001 to 1% of the perfume compositions, most preferably from 0.01 to 0.7%.

Optional Ingredients

The compositions of the present invention may also comprise optional ingredients for example diamine, additional surfactants, solvents, polymeric suds stabiliser, enzymes, builder, perfume, chelating agent and mixtures thereof.

All parts, percentages and ratios used herein are expressed as percent weight unless otherwise specified. All documents cited are, in relevant part, incorporated herein by reference.

Solvent

The present compositions may preferably comprise a solvent. Suitable solvents include diols polymeric glycols and mixtures of both diols and polymeric glycols. Diols suitable for use in the present invention have the following formula:

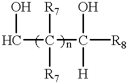

wherein n=0-3, R₇=H, methyl or ethyl; and R₈=H, methyl, ethyl, propyl, isopropyl, butyl and isoubutyl. Preferred diols include propylene glycol, 1,2 hexanediol, 2-ethyl-1,3-hexanediol and 2,2,4-trimethyl-1,3-pentanediol. When diols are present, the present compositions will comprise at least 0.5%, more preferably at least 1%, even more preferably still, at least 3% by weight of the composition of diols. The composition will also preferably contain no more than 20%, more preferably no more than 10%, even more preferably, no more than 6% by weight of the composition of diols.

Polymeric glycols, which comprise ethylene oxide (EO) and propylene oxide (PO) groups may also be included in the present invention. These materials are formed by adding blocks of ethylene oxide moieties to the ends of polypropylene glycol chains. Polymeric gycols suitable for use in the present invention are of the following formula:

(PO)ₓ(EO)_{y}H

wherein x+y is from 17 to 68, and x/(x+y) is from 0.25 to 1.0. A preferred polymeric glycol is a polyproylene glycol (corresponding to when y≈0) having an average molecular weight of between 1000 to 5000, more preferably between 2000 to 4000, most preferably 2000 to 3000.

When polymeric glycols are present the present liquid detergent compositions will contain at least 0.25%, more preferably at least 0.5%, even more preferably still, at least 0.75% by weight of the composition of polymeric glycols.

The composition will also preferably contain no more than 5%, more preferably no more than 3%, even more preferably, no more than 2% by weight of the composition.

To insure satisfactory physical stability, whenever polymeric glycols are added to a liquid dishwashing composition, it may be necessary to also include either a diol and/or an alkali metal inorganic salt, such as sodium chloride. Suitable amounts of diols to provide physical stability are in the amounts in the ranges found above, while a suitable amount of an alkali metal inorganic salt is at least 0.1% and less than 1.5%, preferably less than 0.8% by weight of the composition.

As discussed above, the addition of diols can improve the physical and enzymatic stability of a liquid dishwashing composition.

Other suitable solvents include lower alkanols, diols, other polyols, ethers, amines, and the like may be used in the present invention. Particularly preferred are the C1-C4 alkanols.

Suitable solvents for use herein include ethers and diethers having from 4 to 14 carbon atoms, preferably from 6 to 12 carbon atoms, and more preferably from 8 to 10 carbon atoms. Also other suitable solvents are glycols or alkoxylated glycols, alkoxylated aromatic alcohols, aromatic alcohols, aliphatic branched alcohols, alkoxylated aliphatic branched alcohols, alkoxylated linear C1-C5 alcohols, linear C1-C5 alcohols, C8-C14 alkyl and cycloalkyl hydrocarbons and halohydrocarbons, C6-C16 glycol ethers and mixtures thereof.

Besides propylene glycol,polypropylene glycol and the diols illustrated above, other glycols according to the formula: HO—CR1R2—OH wherein R1 and R2 are independently H or a C2-C10 saturated or unsaturated aliphatic hydrocarbon chain and/or cyclic are suitable and can be used herein. One such suitable glycol is dodecaneglycol.

Suitable alkoxylated glycols which can be used herein are according to the formula

R—(A)ₙ—R¹—OH

wherein R is H, OH, a linear saturated or unsaturated alkyl of from 1 to 20 carbon atoms, preferably from 2 to 15 and more preferably from 2 to 10, wherein R¹ is H or a linear saturated or unsaturated alkyl of from 1 to 20 carbon atoms, preferably from 2 to 15 and more preferably from 2 to 10, and A is an alkoxy group preferably ethoxy, methoxy, and/or propoxy and n is from 1 to 5, preferably 1 to 2. Suitable alkoxylated glycols to be used herein are methoxy octadecanol and/or ethoxyethoxyethanol.

Suitable aromatic alcohols which can be used herein are according to the formula R—OH wherein R is an alkyl substituted or non-alkyl substituted aryl group of from 1 to 20 carbon atoms, preferably from 1 to 15 and more preferably from 1 to 10. For example a suitable aromatic alcohol to be used herein is benzyl alcohol.

Suitable aliphatic branched alcohols which can be used herein are according to the formula R—OH wherein R is a branched saturated or unsaturated alkyl group of from 1 to 20 carbon atoms, preferably from 2 to 15 and more preferably from 5 to 12. Particularly suitable aliphatic branched alcohols to be used herein include 2-ethylbutanol and/or 2-methylbutanol.

Suitable alkoxylated aliphatic branched alcohols which can be used herein are according to the formula R(A)n—OH wherein R is a branched saturated or unsaturated alkyl group of from 1 to 20 carbon atoms, preferably from 2 to 15 and more preferably from 5 to 12, wherein A is an alkoxy group preferably butoxy, propoxy and/or ethoxy, and n is an integer of from 1 to 5, preferably 1 to 2. Suitable alkoxylated aliphatic branched alcohols include 1-methylpropoxyethanol and/or 2-methylbutoxyethanol.

Suitable linear C1-C5 alcohols which can be used herein are according to the formula R—OH wherein R is a linear saturated or unsaturated alkyl group of from 1 to 5 carbon atoms, preferably from 2 to 4. Suitable linear C₁-C₅ alcohols are methanol, ethanol, propanol or mixtures thereof.

Other suitable solvents include, but are not limited to, butyl diglycol ether (BDGE), butyltriglycol ether, ter amilic alcohol and the like. Particularly preferred solvents which can be used herein are butoxy propoxy propanol, butyl diglycol ether, benzyl alcohol, butoxypropanol, ethanol, methanol, isopropanol and mixtures thereof.

Other suitable solvents for use herein include propylene glycol derivatives such as n-butoxypropanol or n-butoxypropoxypropanol, water-soluble CARBITOL R solvents or water-soluble CELLOSOLVE R solvents; water-soluble CARBITOL R solvents are compounds of the 2-(2-alkoxyethoxy)ethanol class wherein the alkoxy group is derived from ethyl, propyl or butyl; a preferred water-soluble carbitol is 2-(2-butoxyethoxy)ethanol also known as butyl carbitol. Water-soluble CELLOSOLVE R solvents are compounds of the 2-alkoxyethoxy ethanol class, with 2-butoxyethoxyethanol being preferred. Other suitable solvents include benzyl alcohol, and diols such as 2-ethyl-1,3-hexanediol and 2,2,4-trimethyl-1,3-pentanediol and mixtures thereof. Some preferred solvents for use herein are n-butoxypropoxypropanol, BUTYL CARBITOL 0 and mixtures thereof.

The solvents can also be selected from the group of compounds comprising ether derivatives of mono-, di- and tri-ethylene glycol, butylene glycol ethers, and mixtures thereof. The molecular weights of these solvents are preferably less than 350, more preferably between 100 and 300, even more preferably between 115 and 250. Examples of preferred solvents include, for example, mono-ethylene glycol n-hexyl ether, mono-propylene glycol n-butyl ether, and tri-propylene glycol methyl ether. Ethylene glycol and propylene glycol ethers are commercially available from the Dow Chemical Company under the tradename “Dowanol” and from the Arco Chemical Company under the tradename “Arcosolv”. Other preferred solvents including mono- and di-ethylene glycol n-hexyl ether are available from the Union Carbide company.

When present the composition will preferably contain at least 0.01%, more preferably at least 0.5%, even more preferably still, at least 1% by weight of the composition of solvent. The composition will also preferably contain no more than 20%, more preferably no more than 10%, even more preferably, no more than 8% by weight of the composition of solvent.

These solvents may be used in conjunction with an aqueous liquid carrier, such as water, or they may be used without any aqueous liquid carrier being present. Solvents are broadly defined as compounds that are liquid at temperatures of 20° C.-25° C. and which are not considered to be surfactants. One of the distinguishing features is that solvents tend to exist as discrete entities rather than as broad mixtures of compounds. Examples of suitable solvents for the present invention include ethanol, propanol, isopropanol, 2-methyl pyrrolidinone, benzyl alcohol and morpholine n-oxide. Preferred among these solvents are ethanol and isopropanol.

Diamines

Another optional although preferred ingredient of the compositions according to the present invention is a diamine. In the context of a hand dishwashing composition, the “usage levels” of such diamine in the compositions herein can vary depending not only on the type and severity of the soils and stains, but also on the wash water temperature, the volume of wash water and the length of time the dishware is contacted with the wash water.

Since the habits and practices of the users of detergent compositions show considerable variation, the composition will preferably contain at least 0.1%, more preferably at least 0.2%, even more preferably, at least 0.25%, even more preferably still, at least 0.5% by weight of said composition of diamine. The composition will also preferably contain no more than 15%, more preferably no more than 10%, even more preferably, no more than 6%, even more preferably, no more than 5%, even more preferably stille, no more than abot 1.5% by weight of said composition of diamine.

It is preferred that the diamines used in the present invention are substantially free from impurities. That is, by “substantially free” it is meant that the diamines are over 95% pure, i.e., preferably 97%, more preferably 99%, still more preferably 99.5%, free of impurities. Examples of impurities which may be present in commercially supplied diamines include 2-Methyl-1,3-diaminobutane and alkylhydropyrimidine. Further, it is believed that the diamines should be free of oxidation reactants to avoid diamine degradation and ammonia formation.

Preferred organic diamines are those in which pK1 and pK2 are in the range of 8.0 to 11.5, preferably in the range of 8.4 to 11, even more preferably from 8.6 to 10.75. Preferred materials for performance and supply considerations are 1,3-bis(methylamine)-cyclohexane (pKa=10 to 10.5), 1,3 propane diamine (pK1=10.5; pK2=8.8), 1,6 hexane diamine (pK1=11; pK2=10), 1,3 pentane diamine (Dytek EP) (pK1=10.5; pK2=8.9), 2-methyl 1,5 pentane diamine (Dytek A) (pK1=11.2; pK2=10.0). Other preferred materials are the primary/primary diamines with alkylene spacers ranging from C4 to C8. In general, it is believed that primary diamines are preferred over secondary and tertiary diamines.

Definition of pK1 and pK2—As used herein, “pKa1” and “pKa2” are quantities of a type collectively known to those skilled in the art as “pKa” pKa is used herein in the same manner as is commonly known to people skilled in the art of chemistry. Values referenced herein can be obtained from literature, such as from “Critical Stability Constants: Volume 2, Amines” by Smith and Martel, Plenum Press, NY and London, 1975. Additional information on pKa's can be obtained from relevant company literature, such as information supplied by Dupont, a supplier of diamines.

As a working definition herein, the pKa of the diamines is specified in an all-aqueous solution at 25° C. and for an ionic strength between 0.1 to 0.5 M. The pKa is an equilibrium constant which can change with temperature and ionic strength; thus, values reported in the literature are sometimes not in agreement depending on the measurement method and conditions. To eliminate ambiguity, the relevant conditions and/or references used for pka's of this invention are as defined herein or in “Critical Stability Constants: Volume 2, Amines”. One typical method of measurement is the potentiometric titration of the acid with sodium hydroxide and determination of the pKa by suitable methods as described and referenced in “The Chemist's Ready Reference Handbook” by Shugar and Dean, McGraw Hill, NY, 1990.

It has been determined that substituents and structural modifications that lower pK1 and pK2 to below 8.0 are undesirable and cause losses in performance. This can include substitutions that lead to ethoxylated diamines, hydroxy ethyl substituted diamines, diamines with oxygen in the beta (and less so gamma) position to the nitrogen in the spacer group (e.g., Jeffamine EDR 148). In addition, materials based on ethylene diamine are unsuitable.

The diamines useful herein can be defined by the following structure:

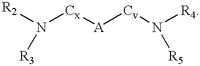

wherein R₂₋₅ are independently selected from H, methyl, —CH₃CH₂, and ethylene oxides; Cₓ and Cᵥ are independently selected from methylene groups or branched alkyl groups where x+y is from 3 to 6; and A is optionally present and is selected from electron donating or withdrawing moieties chosen to adjust the diamine pKa's to the desired range. If A is present, then x and y must both be 1 or greater.

Examples of preferred diamines can be found in the copending provisional patent application of Phillip Kyle Vinson et al., entitled “Dishwashing Detergent Compositions Containing Organic Diamines for Improved Grease Cleaning, Sudsing, Low Temperature Stability and Dissolution”, having P & G Case No. 7167P, application Ser. No. 60/087,693, and filed on Jun. 2, 1998, which is hereby incorporated by reference.

Carboxylic Acid

The compositions according to the present invention may comprise a linear or cyclic carboxylic acid or salt thereof. Where the acid or salt thereof is present and is linear, it preferably comprises from 1 to 6 carbon atoms whereas where the acid is cyclic, it preferably comprises greater than 3 carbon atoms. The linear or cyclic carbon-containing chain of the carboxylic acid or salt thereof may be substituted with a substituent group selected from the group consisting of hydroxyl, ester, ether, aliphatic groups having from 1 to 6, more preferably 1 to 4 carbon atoms and mixtures thereof

The carboxylic acids or salts thereof preferably have a pka1 of less than 7, more preferably from 1 to 3. The carboxylic acid and salts thereof may comprise one or two or more carboxylic groups.

Suitable carboxylic acids or salts thereof are those having the general formula:

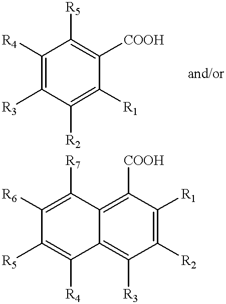

wherein R₁, R₂, R₃, R₄, R₅, R₆, R₇ are selected from the group consisting of alkyl chain having from 1 to 3 carbon atoms, hydroxy group, hydrogen, ester group, carboxylic acid group with the proviso that no more than 3 carboxylic acid groups are present.

Preferred carboxylic acids are those selected from the group consisting of salicylic acid, maleic acid, acetyl salicylic acid, 3 methyl salicylic acid, 4 hydroxy isophthalic acid, dihydroxyfumaric acid, 1,2,4 benzene tricarboxylic acid, pentanoic acid and salts thereof and mixtures thereof. Where the carboxylic acid exists in the salt form, the cation of the salt is preferably selected from alkali metal, alkaline earth metal, monoethanolamine, diethanolamine or triethanolamine and mixtures thereof.

The carboxylic acid or salt thereof is preferably present at the level of from 0.1% to 5%, more preferably from 0.2% to 1% and most preferably from 0.25% to 0.5%.

As described above the compositions herein are used to provide improved rinse feel as defined below. The Applicants believe to have found that the presence of anionic surfactants, especially when present in higher amounts in the region of 15-35% by weight of the composition, results in the composition imparting a slippery feel to the hands of the user and the dishware. This feeling of slipperiness is reduced when using the compositions as defined herein i.e. the rinse feel becomes draggy. Although not wishing to be bound by theory it is the Applicants belief that the carboxylic acid provides this benefit. Moreover, it is also believed that formulating the composition with anionic surfactant, the carbon chain of which is branched such that the anionic surfactant comprises on average greater than 30% branching, can also provide or improve the rinse feel.

By the term “rinse feel” it is meant the feel of the hands of the user or the feel of the user hands on the dishware, especially when rinsing the dish with water. The type of rinse feel provided by a composition can be described as either “slippery”, where it is perceived that composition and rinse water are more difficult to remove from the surface as opposed to “draggy” where it is perceived that the composition and rinse water are comparatively easier to remove. “Draggy” rinse feel can equally be detected as an increase in friction between skin of the user or between skin and dishware. By the term “improved rinse feel” it is meant herein that the compositions of the present invention provide a comparatively more “draggy” rinse feel when compared with a composition not comprising either branched anionic surfactant or carboxylic acid as defined herein or mixtures thereof.

Surfactant

The compositions of the present invention may optionally although preferably comprise a surfactant. Surfactants may be selected from the group consisting of amphoteric, zwitterionic, nonionic other anionic, cationic surfactants and mixtures thereof.

Amphoteric surfactants are preferred additional surfactants. The amphoteric surfactants useful in the present invention are preferably selected from amine oxide surfactants. Amine oxides are semi-polar nonionic surfactants and include water-soluble amine oxides containing one alkyl moiety of from 10 to 18 carbon atoms and 2 moieties selected from the group consisting of alkyl groups and hydroxyalkyl groups containing from 1 to 3 carbon atoms; water-soluble phosphine oxides containing one alkyl moiety of from 10 to 18 carbon atoms and 2 moieties selected from the group consisting of alkyl groups and hydroxyalkyl groups containing from 1 to 3 carbon atoms; and water-soluble sulfoxides containing one alkyl moiety of from 10 to 18 carbon atoms and a moiety selected from the group consisting of alkyl and hydroxyalkyl moieties of from 1 to 3 carbon atoms.

Semi-polar nonionic detergent surfactants include the amine oxide surfactants having the formula

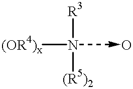

wherein R³ is an alkyl, hydroxyalkyl, or alkyl phenyl group or mixtures thereof containing from 8 to 22 carbon atoms; R⁴ is an alkylene or hydroxyalkylene group containing from 2 to 3 carbon atoms or mixtures thereof; x is from 0 to 3; and each R⁵ is an alkyl or hydroxyalkyl group containing from 1 to 3 carbon atoms or a polyethylene oxide group containing from 1 to 3 ethylene oxide groups. The R⁵ groups can be attached to each other, e.g., through an oxygen or nitrogen atom, to form a ring structure.

These amine oxide surfactants in particular include C₁₀-C₁₈ alkyl dimethyl amine oxides and C₈-C₁₂ alkoxy ethyl dihydroxy ethyl amine oxides.

Also suitable are amine oxides such as propyl amine oxides, represented by the formula:

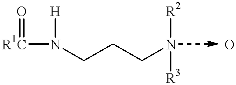

wherein R₁ is an alkyl, 2-hydroxyalkyl, 3-hydroxyalkyl, or 3-alkoxy-2-hydroxypropyl radical in which the alkyl and alkoxy, respectively, contain from 8 to 18 carbon atoms, R₂ and R₃ are each methyl, ethyl, propyl, isopropyl, 2-hydroxyethyl, 2-hydroxypropyl, or 3-hydroxypropyl and n is from 0 to 10.

A further suitable species of amine oxide semi-polar surface active agents comprise compounds and mixtures of compounds having the formula:

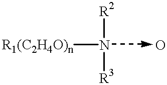

wherein R₁ is an alkyl, 2-hydroxyalkyl, 3-hydroxyalkyl, or 3-alkoxy-2-hydroxypropyl radical in which the alkyl and alkoxy, respectively, contain from 8 to 18 carbon atoms, R₂ and R₃ are each methyl, ethyl, propyl, isopropyl, 2-hydroxyethyl, 2-hydroxypropyl, or 3-hydroxypropyl and n is from 0 to 10. Particularly preferred are amine oxides of the formula:

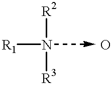

wherein R₁ is a C₁₀₋₁₄ alkyl and R₂ and R₃ are methyl or ethyl. Because they are low-foaming it may also be desirable to use long chain amine oxide surfactants which are more fully described in U.S. Pat. Nos. 4,316,824 (Pancheri), 5,075,501 and 5,071,594, incorporated herein by reference.

Other suitable, non-limiting examples of amphoteric detergent surfactants that are useful in the present invention include amido propyl betaines and derivatives of aliphatic or heterocyclic secondary and ternary amines in which the aliphatic moiety can be straight chain or branched and wherein one of the aliphatic substituents contains from 8 to 24 carbon atoms and at least one aliphatic substituent contains an anionic water-solubilizing group.

Further examples of suitable amphoteric surfactants are given in “Surface Active Agents and Detergents” (Vol. I and II by Schwartz, Perry and Berch), hereby incorporated by reference.

Preferably the amphoteric surfactant where present, is present in the composition in an effective amount, more preferably from 0.1% to 20%, even more preferably 0.1% to 15%, even more preferably still from 0.5% to 10%, by weight.

Suitable nonionic detergent surfactants are generally disclosed in U.S. Pat. No. 3,929,678, Laughlin et al., issued Dec. 30, 1975, at column 13, line 14 through column 16, line 6, incorporated herein by reference.

The condensation products of aliphatic alcohols with from 1 to 25 moles of ethylene oxide. The alkyl chain of the aliphatic alcohol can either be straight or branched, primary or secondary, and generally contains from 8 to 22 carbon atoms. Particularly preferred are the condensation products of alcohols having an alkyl group containing from 10 to 20 carbon atoms with from 2 to 18 moles of ethylene oxide per mole of alcohol. Examples of commercially available nonionic surfactants of this type include Tergitol® 15-S-9 (the condensation product of C₁₁-C₁₅ linear secondary alcohol with 9 moles ethylene oxide), Tergitol® 24-L-6 NMW (the condensation product of C₁₂-C₁₄ primary alcohol with 6 moles ethylene oxide with a narrow molecular weight distribution), both marketed by Union Carbide Corporation; Neodol® 45-9 (the condensation product of C₁₄-C₁₅ linear alcohol with 9 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 45-4 (the condensation product of C₁₄-C₁₅ linear alcohol with 4 moles of ethylene oxide), marketed by Shell Chemical Company, and Kyro® EOB (the condensation product of C₁₃-C₁₅ alcohol with 9 moles ethylene oxide), marketed by The Procter & Gamble Company. Other commercially available nonionic surfactants include Dobanol 91-8® marketed by Shell Chemical Co. and Genapol UD-080® marketed by Hoechst. This category of nonionic surfactant is referred to generally as “alkyl ethoxylates.”

The preferred alkylpolyglycosides have the formula

R²O(CₙH₂ₙO)ₜ(glycosyl)ₓ

wherein R² is selected from the group consisting of alkyl, alkyl-phenyl, hydroxyalkyl, hydroxyalkylphenyl, and mixtures thereof in which the alkyl groups contain from 10 to 18, preferably from 12 to 14, carbon atoms; n is 2 or 3, preferably 2; t is from 0 to 10, preferably 0; and x is from 1.3 to 10, preferably from 1.3 to 3, most preferably from 1.3 to 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position.

Fatty acid amide surfactants having the formula:

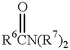

wherein R⁶ is an alkyl group containing from 7 to 21 (preferably from 9 to 17) carbon atoms and each R⁷ is selected from the group consisting of hydrogen, C₁-C₄ alkyl, C₁-C₄ hydroxyalkyl, and —(C²H₄O)ₓH where x varies from 1 to 3.

Preferred amides are C₈-C₂₀ ammonia amides, monoethanolamides, diethanolamides, and isopropanolamides.

Preferably the nonionic surfactant, when present in the composition, is present in an effective amount, more preferably from 0.1% to 20%, even more preferably 0.1% to 15%, even more preferably still from 0.5% to 10%, by weight.

The detergent compositions hereof may also contain an effective amount of polyhydroxy fatty acid amide surfactant. By “effective amount” is meant that the formulator of the composition can select an amount of polyhydroxy fatty acid amide to be incorporated into the compositions that will improve the cleaning performance of the detergent composition. In general, for conventional levels, the incorporation of 1%, by weight, polyhydroxy fatty acid amide will enhance cleaning performance.

Where present, the detergent compositions may comprise 1% weight basis, polyhydroxy fatty acid amide surfactant, preferably from 3% to 30%, of the polyhydroxy fatty acid amide. The polyhydroxy fatty acid amide surfactant component comprises compounds of the structural formula:

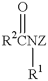

wherein: R¹ is H, C₁-C₄ hydrocarbyl, 2-hydroxy ethyl, 2-hydroxy propyl, or a mixture thereof, preferably C₁-C₄ alkyl, more preferably C₁ or C₂ alkyl, most preferably C₁ alkyl (i.e., methyl); and R² is a C₅-C₃₁ hydrocarbyl, preferably straight chain C₇-C₁₉ alkyl or alkenyl, more preferably straight chain C₉-C₁₇ alkyl or alkenyl, most preferably straight chain C₁₁-C₁₅ alkyl or alkenyl, or mixtures thereof; and Z is a polyhydroxyhydrocarbyl having a linear hydrocarbyl chain with at least 3 hydroxyls directly connected to the chain, or an alkoxylated derivative (preferably ethoxylated or propoxylated) thereof. Z preferably will be derived from a reducing sugar in a reductive amination reaction; more preferably Z will be a glycityl. Suitable reducing sugars include glucose, fructose, maltose, lactose, galactose, mannose, and xylose. As raw materials, high dextrose corn syrup, high fructose corn syrup, and high maltose corn syrup can be utilized as well as the individual sugars listed above. These corn syrups may yield a mix of sugar components for Z. It should be understood that it is by no means intended to exclude other suitable raw materials. Z preferably will be selected from the group consisting of —CH₂—(CHOH)ₙ—CH₂OH, —CH(CH₂OH)—(CHOH)ₙ₋₁—CH₂OH, —CH₂—(CHOH)₂(CHOR′)(CHOH)—CH₂OH, and alkoxylated derivatives thereof, where n is an integer from 3 to 5, inclusive, and R′ is H or a cyclic or aliphatic monosaccharide. Most preferred are glycityls wherein n is 4, particularly —CH₂—(CHOH)₄—CH₂OH.

R′ can be, for example, N-methyl, N-ethyl, N-propyl, N-isopropyl, N-butyl, N-2-hydroxy ethyl, or N-2-hydroxy propyl.

R²—CO—N< can be, for example, cocamide, stearamide, oleamide, lauramide, myristamide, capricamide, palmitamide, tallowamide, etc.

Z can be 1-deoxyglucityl, 2-deoxyfructityl, 1-deoxymaltityl, 1-deoxylactityl, 1-deoxygalactityl, 1-deoxymannityl, 1-deoxymaltotriotityl, etc.

Suitable anionic surfactants for use in the compositions herein include water-soluble salts or acids of the formula ROSO₃M wherein R preferably is a C₆-C₂₀ linear or branched hydrocarbyl, preferably an alkyl or hydroxyalkyl having a C₁₀-C₂₀ alkyl component, more preferably a C₁₀-C₁₄ alkyl or hydroxyalkyl, and M is H or a cation, e.g., an alkali metal cation or ammonium or substituted ammonium, but preferably sodium.

Other suitable anionic surfactants for use herein are water-soluble salts or acids of the formula RO(A)ₘSO₃M wherein R is an unsubstituted linear or branched C₆-C₂₀ alkyl or hydroxyalkyl group having a C₁₀-C₂₀ alkyl component, preferably a C₁₂-C₂₀ alkyl or hydroxyalkyl, more preferably C₁₂-C₁₄ alkyl or hydroxyalkyl, A is an ethoxy or propoxy unit, m is greater than zero, typically between 0.5 and 5, more preferably between 0.5 and 2, and M is H or a cation which can be, for example, a metal cation, ammonium or substituted-ammonium cation. Alkyl ethoxylated sulfates as well as alkyl propoxylated sulfates are contemplated herein. Exemplary surfactants are C₁₀-C₁₄ alkyl polyethoxylate (1.0) sulfate, C₁₀-C₁₄ polyethoxylate (1.0) sulfate, C₁₀-C₁₄ alkyl polyethoxylate (2.25) sulfate, C₁₀-C₁₄ polyethoxylate (2.25) sulfate, C₁₀-C₁₄ alkyl polyethoxylate (3.0) sulfate, C₁₀-C₁₄ polyethoxylate (3.0) sulfate, and C₁₀-C₁₄ alkyl polyethoxylate (4.0) sulfate, C₁₀-C₁₈ polyethoxylate (4.0) sulfate. In a preferred embodiment the anionic surfactant is a mixture of alkoxylated, preferably ethoxylated and non-alkoxylated sulfate surfactants. In such a preferred embodiment the preferred average degree of alkoxylation is from 0.4 to 0.8.

Other particularly suitable anionic surfactants for use herein are alkyl sulphonates including water-soluble salts or acids of the formula RSO₃M wherein R is a C₆-C₂₀ linear or branched, saturated or unsaturated alkyl group, preferably a C₁₀-C₂₀ alkyl group and more preferably a C₁₀-C₁₄ alkyl group, and M is H or a cation, e.g., an alkali metal cation (e.g., sodium, potassium, lithium), or ammonium or substituted ammonium (e.g., methyl-, dimethyl-, and trimethyl ammonium cations and quaternary ammonium cations, such as tetramethyl-ammonium and dimethyl piperdinium cations and quaternary ammonium cations derived from alkylamines such as ethylamine, diethylamine, triethylamine, and mixtures thereof, and the like).

Suitable alkyl aryl sulphonates for use herein include water-soluble salts or acids of the formula RSO₃M wherein R is an aryl, preferably a benzyl, substituted by a C₆-C₂₀ linear or branched saturated or unsaturated alkyl group, preferably a C₁₂-C₁₆ alkyl group and more preferably a C₁₀-C₁₄ alkyl group, and M is H or a cation, e.g., an alkali metal cation (e.g., sodium, potassium, lithium, calcium, magnesium etc) or ammonium or substituted ammonium (e.g., methyl-, dimethyl-, and trimethyl ammonium cations and quaternary ammonium cations, such as tetramethyl-ammonium and dimethyl piperdinium cations and quaternary ammonium cations derived from alkylamines such as ethylamine, diethylamine, triethylamine, and mixtures thereof, and the like).

In a further preferred embodiment the carbon chain of the anionic surfactant comprises alkyl, preferably C1-4 alkyl branching units. The average percentage branching of the anionic surfactant is greater than 30%, more preferably from 35% to 80% and most preferably from 40% to 60%. Such average percentage of branching can be achieved by formulating the composition with one or more anionic surfactants all of which are preferably greater than 30% branched, more preferably from 35% to 80% and most preferably from 40% to 60%. Alternatively and more preferably, the composition may comprise a combination of branched anionic surfactant and linear anionic surfactant such that on average the percentage of branching of the total anionic surfactant combination is greater than 30%, more preferably from 35% to 80% and most preferably from 40% to 60%.

The anionic surfactant comprising on average less than 4 moles of alkoxy groups is preferably present at a level of at least 10%, more preferably from 15% to 40% and most preferably from 20% to 35% by weight of the total composition. The anionic surfactant comprising on average at least 4 moles of alkoxy groups is preferably present at a level of at least 20%, more preferably from 25% to 35% by weight of the total composition.

Other additional anionic surfactants useful for detersive purposes can also be used herein. These can include salts (iancluding, for example, sodium, potassium, ammonium, and substituted ammonium salts such as mono-, di- and triethanolamine salts) of soap, C₈-C₂₄ olefinsulfonates, sulfonated polycarboxylic acids prepared by sulfonation of the pyrolyzed product of alkaline earth metal citrates, e.g., as described in British patent specification No. 1,082,179, C₈-C₂₄ alkylpolyglycolethersulfates (containing up to 10 moles of ethylene oxide); alkyl ester sulfonates such as C₁₄₋₁₆ methyl ester sulfonates; acyl glycerol sulfonates, fatty oleyl glycerol sulfates, alkyl phenol ethylene oxide ether sulfates, paraffin sulfonates, alkyl phosphates, isethionates such as the acyl isethionates, N-acyl taurates, alkyl succinamates and sulfosuccinates, monoesters of sulfosuccinate (especially saturated and unsaturated C₁₂-C₁₈ monoesters) diesters of sulfosuccinate (especially saturated and unsaturated C₆-C₁₄ diesters), sulfates of alkylpolysaccharides such as the sulfates of alkylpolyglucoside (the nonionic nonsulfated compounds being described below), branched primary alkyl sulfates, alkyl polyethoxy carboxylates such as those of the formula RO(CH₂CH₂O)ₖCH₂COO—M⁺ wherein R is a C₈-C₂₂ alkyl, k is an integer from 0 to 10, and M is a soluble salt-forming cation. Resin acids and hydrogenated resin acids are also suitable, such as rosin, hydrogenated rosin, and resin acids and hydrogenated resin acids present in or derived from tall oil. Further examples are given in “Surface Active Agents and Detergents” (Vol. I and II by Schwartz, Perry and Berch). A variety of such surfactants are also generally disclosed in U.S. Pat. No. 3,929,678, issued Dec. 30, 1975, to Laughlin, et al. at Column 23, line 58 through Column 29, line 23 (herein incorporated by reference).

Other particularly suitable anionic surfactants for use herein are alkyl carboxylates and alkyl alkoxycarboxylates having from 4 to 24 carbon atoms in the alkyl chain, preferably from 8 to 18 and more preferably from 8 to 16, wherein the alkoxy is propoxy and/or ethoxy and preferably is ethoxy at an alkoxylation degree of from 0.5 to 20, preferably from 5 to 15. Preferred alkylalkoxycarboxylate for use herein is sodium laureth 11 carboxylate (i.e., RO(C₂H₄O)₁₀—CH₂COONa, with R=C12-C14) commercially available under the name Akyposoft® 100NV from Kao Chemical Gbmh.

The particular surfactants used can therefore vary widely depending upon the particular end-use envisioned. Suitable additional surfactants are described in detail in the copending provisional patent application of Chandrika Kasturi et al., entitled “Liquid Detergent Compositions Comprising Polymeric Suds Enhancers”, having P & G Case No. 6938P, application Ser. No. 60/066,344, incorporated above.

Polymeric Suds Stabilizer

The compositions of the present invention may optionally contain a polymeric suds stabilizer. These polymeric suds stabilizers provide extended suds volume and suds duration without sacrificing the grease cutting ability of the liquid detergent compositions. These polymeric suds stabilizers are selected from:

i) homopolymers of (N,N-dialkylamino)alkyl acrylate esters having the formula:

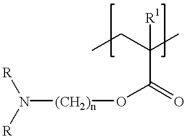

wherein each R is independently hydrogen, C₁-C₈ alkyl, and mixtures thereof, R¹ is hydrogen, C₁-C₆ alkyl, and mixtures thereof, n is from 2 to 6; and

ii) copolymers of (i) and

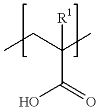

wherein R¹ is hydrogen, C1-C6 alkyl, and mixtures thereof, provided that the ratio of (ii) to (i) is from 2 to 1 to 1 to 2; The molecular weight of the polymeric suds boosters, determined via conventional gel permeation chromatography, is from 1,000 to 2,000,000, preferably from 5,000 to 1,000,000, more preferably from 10,000 to 750,000, more preferably from 20,000 to 500,000, even more preferably from 35,000 to 200,000. The polymeric suds stabilizer can optionally be present in the form of a salt, either an inorganic or organic salt, for example the citrate, sulfate, or nitrate salt of (N,N-dimethylamino)alkyl acrylate ester.

One preferred polymeric suds stabilizer is (N,N-dimethylamino)alkyl acrylate esters, namely

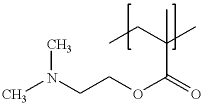

When present in the compositions, the polymeric suds booster may be present in the composition from 0.01% to 15%, preferably from 0.05% to 10%, more preferably from 0.1% to 5%, by weight.

Builder

The compositions according to the present invention may further comprise a builder system. Because builders such as citric acid and citrates impair the stability of enzymes in LDL compositions, it is desirable to include reduce the amounts or completely remove the builder salts normally utilized in LDL compositions incorporating propylene glycol as a builder. When a detergent composition includes propylene glycol solvent as a part or a whole of the detergent's carrier, enzymes are more stable and smaller amounts or no builder salts are needed.

If it is desirable to use a builder, then any conventional builder system is suitable for use herein including aluminosilicate materials, silicates, polycarboxylates and fatty acids, materials such as ethylene-diamine tetraacetate-metal ion sequestrants such as aminopolyphosphonates, particularly ethylenediamine tetramethylene phosphonic acid and diethylene triamine pentamethylene-phosphonic acid. Though less preferred for obvious environmental reasons, phosphate builders can also be used herein.

Suitable polycarboxylates builders for use herein include citric acid, preferably in the form of a water-soluble salt, derivatives of succinic acid of the formula R—CH(COOH)CH₂(COOH) wherein R is C₁₀₋₂₀ alkyl or alkenyl, preferably C₁₂-C₁₆, or wherein R can be substituted with hydroxyl, sulfo sulfoxyl or sulfone substituents. Specific examples include lauryl succinate, myristyl succinate, palmityl succinate 2-dodecenylsuccinate, 2-tetradecenyl succinate. Succinate builders are preferably used in the form of their water-soluble salts, including sodium, potassium, ammonium and alkanolammonium salts.

Other suitable polycarboxylates are oxodisuccinates and mixtures of tartrate monosuccinic and tartrate disuccinic acid such as described in U.S. Pat. No. 4,663,071.

Especially for the liquid execution herein, suitable fatty acid builders for use herein are saturated or unsaturated C₁₀₋₁₈ fatty acids, as well as the corresponding soaps. Preferred saturated species have from 12 to 16 carbon atoms in the alkyl chain. The preferred unsaturated fatty acid is oleic acid. Other preferred builder system for liquid compositions is based on dodecenyl succinic acid and citric acid.

If detergency builder salts are included, they will be included in amounts of from 0.5% to 50% by weight of the composition preferably from 5% to 30% and most usually from 5% to 25% by weight.

Enzymes

Detergent compositions of the present invention may further comprise one or more enzymes which provide cleaning performance benefits. Said enzymes include enzymes selected from cellulases, hemicellulases, peroxidases, proteases, gluco-amylases, amylases, lipases, cutinases, pectinases, xylanases, reductases, oxidases, phenoloxidases, lipoxygenases, ligninases, pullulanases, tannases, pentosanases, malanases, β-glucanases, arabinosidases or mixtures thereof. A preferred combination is a detergent composition having a cocktail of conventional applicable enzymes like protease, amylase, lipase, cutinase and/or cellulase. Enzymes when present in the compositions, at from 0.0001% to 5% of active enzyme by weight of the detergent composition. Preferred proteolytic enzymes, then, are selected from the group consisting of Alcalase® (Novo Industri A/S), BPN′, Protease A and Protease B (Genencor), and mixtures thereof. Protease B is most preferred. Preferred amylase enzymes include TERMAMYL®, DURAMYL® and the amylase enzymes those described in WO 9418314 to Genencor International and WO 9402597 to Novo.

Further non-limiting examples of suitable and preferred enzymes are disclosed in the copending application: “Dishwashing Detergent Compositions Containing Organic Diamines for Improved Grease Cleaning, Sudsing, Low temperature stability and Dissolution”, having P & G Case No. 7167P and application Ser. No. 60/087,693, which is hereby incorporated by reference.

Because hydrogen peroxide and builders such as citric acid and citrates impair the stability of enzymes in LDL compositions, it is desirable to reduce or eliminate the levels of these compounds in compositions which contain enzymes. Hydrogen peroxide is often found as an impurity in surfactants and surfactant pastes. As such, the preferred level of hydrogen peroxide in the amine oxide or surfactant paste of amine oxide is 0-40 ppm, more preferably 0-15 ppm. Amine impurities in amine oxide and betaines, if present, should be minimized to the levels referred above for hydrogen peroxide.

Magnesium ions

While it is preferred that divalent ions be omitted from LDL compositions prepared according to the present invention, alternate embodiments of the present invention may include magnesium ions.

It is desirable to exclude all divalent ions from the present LDL compositions, because such ions may lead to slower dissolution as well as poor rinsing, and poor low temperature stability properties. Moreover, formulating such divalent ion-containing compositions in alkaline pH matrices may be difficult due to the incompatibility of the divalent ions, particularly magnesium, with hydroxide ions.

Nonetheless, the presence of magnesium ions offers several benefits. Notably, the inclusion of such divalent ions improves the cleaning of greasy soils for various LDL compositions, in particular compositions containing alkyl ethoxy carboxylates and/or polyhydroxy fatty acid amide. This is especially true when the compositions are used in softened water that contains few divalent ions.

But in the present invention, these benefits can be obtained without the inclusion of divalent ions. In particular, improved grease cleaning can be achieved without divalent ions by the inclusion of organic diamines in combination with amphoteric and anionic surfactants in the specific ratios discussed above while enzymes have been shown to improve the skin mildness performance of the present LDL compositions.

If they are to be included in an alternate embodiment of the present LDL compositions, then the magnesium ions are present at an active level of from 0.01% to 1.5%, preferably from 0.015% to 1%, more preferably from 0.025% to 0.5%, by weight. The amount of magnesium ions present in compositions of the invention will be also dependent upon the amount of total surfactant present therein, including the amount of alkyl ethoxy carboxylates and polyhydroxy fatty acid amide.

Preferably, the magnesium ions are added as a hydroxide, chloride, acetate, sulfate, formate, oxide or nitrate salt to the compositions of the present invention. Because during storage, the stability of these compositions becomes poor due to the formation of hydroxide precipitates in the presence of compositions containing moderate concentrations of hydroxide ions, it may be necessary to add certain chelating agents. Suitable chelating agents are discussed further below and in U.S. Pat. No. 5,739,092, issued Apr. 14, 1998, to Ofosu-asante, incorporated herein by reference.

Chelating Agents

The detergent compositions herein may also optionally contain one or more iron and/or manganese chelating agents. Such chelating agents can be selected from the group consisting of amino carboxylates, amino phosphonates, polyfunctionally-substituted aromatic chelating agents and mixtures therein, all as hereinafter defined. Without intending to be bound by theory, it is believed that the benefit of these materials is due in part to their exceptional ability to remove iron and manganese ions from washing solutions by formation of soluble chelates.

Amino carboxylates useful as optional chelating agents include ethylenediaminetetrace-tates, N-hydroxyethylethylenediaminetriacetates, nitrilo-tri-acetates, ethylenediamine tetrapro-prionates, triethylenetetraaminehexacetates, diethylenetriaminepentaacetates, and ethanoldi-glycines, alkali metal, ammonium, and substituted ammonium salts therein and mixtures therein.

Amino phosphonates are also suitable for use as chelating agents in the compositions of the invention when at lease low levels of total phosphorus are permitted in detergent compositions, and include ethylenediaminetetrakis (methylenephosphonates) as DEQUEST. Preferred, these amino phosphonates to not contain alkyl or alkenyl groups with more than 6 carbon atoms.

Polyfunctionally-substituted aromatic chelating agents are also useful in the compositions herein. See U.S. Pat. No. 3,812,044, issued May 21, 1974, to Connor et al. Preferred compounds of this type in acid form are dihydroxydisulfobenzenes such as 1,2-dihydroxy-3,5-disulfobenzene.

A preferred biodegradable chelator for use herein is ethylenediamine disuccinate (“EDDS”), especially the [S,S] isomer as described in U.S. Pat. No. 4,704,233, Nov. 3, 1987, to Hartman and Perkins.

The compositions herein may also contain water-soluble methyl glycine diacetic acid (MGDA) salts (or acid form) as a chelant or co-builder. Similarly, the so called “weak” builders such as citrate can also be used as chelating agents.

If utilized, these chelating agents will generally comprise from 0.00015% to 15% by weight of the detergent compositions herein. More preferably, if utilized, the chelating agents will comprise from 0.0003% to 3.0% by weight of such compositions.

Other Ingredients—The detergent compositions will further preferably comprise one or more detersive adjuncts selected from the following: soil release polymers, polymeric dispersants, polysaccharides, abrasives, bactericides and other antimicrobals, tarnish inhibitors, builders, enzymes, dyes, buffers, antifungal or mildew control agents, insect repellents, perfumes, hydrotropes, thickeners, processing aids, suds boosters, brighteners, anti-corrosive aids, stabilizers antioxidants and chelants. A wide variety of other ingredients useful in detergent compositions can be included in the compositions herein, including other active ingredients, carriers, hydrotropes, antioxidants, processing aids, dyes or pigments, solvents for liquid formulations, solid fillers for bar compositions, etc. If high sudsing is desired, suds boosters such as the C₁₀-C₁₆ alkanolamides can be incorporated into the compositions, typically at 1%-10% levels. The C₁₀-C₁₄ monoethanol and diethanol amides illustrate a typical class of such suds boosters. Use of such suds boosters with high sudsing adjunct surfactants such as the amine oxides, betaines and sultaines noted above is also advantageous.

An antioxidant can be optionally added to the detergent compositions of the present invention. They can be any conventional antioxidant used in detergent compositions, such as 2,6-di-tert-butyl-4-methylphenol (BHT), carbamate, ascorbate, thiosulfate, monoethanolamine(MEA), diethanolamine, triethanolamine, etc. It is preferred that the antioxidant, when present, be present in the composition from 0.001% to 5% by weight.

Various detersive ingredients employed in the present compositions optionally can be further stabilized by absorbing said ingredients onto a porous hydrophobic substrate, then coating said substrate with a hydrophobic coating. Preferably, the detersive ingredient is admixed with a surfactant before being absorbed into the porous substrate. In use, the detersive ingredient is released from the substrate into the aqueous washing liquor, where it performs its intended detersive function.

To illustrate this technique in more detail, a porous hydrophobic silica (trademark SIPERNAT D10, DeGussa) is admixed with a proteolytic enzyme solution containing 3%-5% of C₁₃₋₁₅ ethoxylated alcohol (EO 7) nonionic surfactant. Typically, the enzyme/surfactant solution is 2.5×the weight of silica. The resulting powder is dispersed with stirring in silicone oil (various silicone oil viscosities in the range of 500-12,500 can be used). The resulting silicone oil dispersion is emulsified or otherwise added to the final detergent matrix. By this means, ingredients such as the aforementioned enzymes, bleaches, bleach activators, bleach catalysts, photoactivators, dyes, fluorescers, fabric conditioners and hydrolyzable surfactants can be “protected” for use in detergents, including liquid laundry detergent compositions.

Further, these hand dishwashing detergent embodiments preferably further comprises a hydrotrope. Suitable hydrotropes include sodium, potassium, ammonium or water-soluble substituted ammonium salts of toluene sulfonic acid, naphthalene sulfonic acid, cumene sulfonic acid, xylene sulfonic acid.

Non-Aqueous Liquid Detergents

The manufacture of liquid detergent compositions which comprise a non-aqueous carrier medium can be prepared according to the disclosures of U.S. Pat. Nos. 4,753,570; 4,767,558; 4,772,413; 4,889,652; 4,892,673; GB-A-2,158,838; GB-A-2,195,125; GB-A-2,195,649; U.S. Pat. Nos. 4,988,462; 5,266,233; EP-A-225,654 (Jun. 16, 1987); EP-A-510,762 (Oct. 28, 1992); EP-A-540,089 (May 5, 1993); EP-A-540,090 (May 5, 1993); U.S. Pat. No. 4,615,820; EP-A-565,017 (Oct. 13, 1993); EP-A-030,096 (Jun. 10, 1981), incorporated herein by reference. Such compositions can contain various particulate detersive ingredients stably suspended therein. Such non-aqueous compositions thus comprise a LIQUID PHASE and, optionally but preferably, a SOLID PHASE, all as described in more detail hereinafter and in the cited references.

Process of Cleaning Dishware

The present invention also relates to a process for cleaning dishware. The dishware is contacted with a composition as described above. The composition may be applied to the dishware neat or in dilute form. Thus the dishware may be cleaned singly by applying the composition to the dishware and optionally but preferably subsequently rinsing before drying. Alternatively, the composition can be mixed with water in a suitable vessel, for example a basin, sink or bowl and thus a number of dishes can be cleaned using the same composition and water (dishwater). In a further alternative process the product can be used in dilute form in a suitable vessel as a soaking medium for, typically extremely dirty, dishware. As before the dishware can be optionally, although preferably, rinsed before allowing to dry. Drying make take place passively by allowing for the natural evaporation of water or actively using any suitable drying equipment, for example a cloth or towel.

### EXAMPLES

The following examples of the cleaning composition are in no way meant to be limiting.

Examples 1-6 describe Liqht Duty Liquid dishwashing detergents according to the present invention:

| | Exam- | Exam- | Exam- | Exam- | Exam- | Exam- |
|---|---|---|---|---|---|---|
| | ple | ple | ple | ple | ple | ple |
| | 1 | 2 | 3 | 4 | 5 | 6 |
| AE0.6S¹ | 26.1 | 26.1 | 26.1 | 26.1 | 13.05 | 26.1 |
| Amine oxide² | 6.5 | 6.5 | 6.5 | 6.5 | 3.25 | 5.5 |
| Nonionic³ | — | 3 | 3 | 3 | 1.5 | 3 |
| Suds boosting | 0.2 | 0.2 | 0.2 | 0.2 | 0.1 | 0.2 |
| polymer⁴ | | | | | | |
| Diamine⁵ | 0.5 | 0.5 | 0.5 | 0.5 | 0.25 | 0.5 |
| Sodium | 3.50 | 3.5 | 3.5 | 3.5 | 1.75 | 2.0 |
| cumene | | | | | | |
| sulphonate | | | | | | |
| sodium | — | — | 0.5 | 0.5 | 0.25 | 0.6 |
| chloride | | | | | | |
| propylene | 9.8 | 9.8 | — | 10.0 | 5.0 | — |
| glycol | | | | | | |
| polypropylene | — | — | 1.0 | 1.0 | 0.5 | 1.0 |
| glycol | | | | | | |
| Citrate | 2.6 | 2.6 | — | — | — | — |
| Mg²⁺ | — | — | — | — | — | 0.04 |
| Protease⁶ | — | — | — | 0.015 | 0.075 | — |
| Ethanol | — | — | 7.0 | 0.0 | 0.0 | 7.0 |
| Perfume from | 0.51 | — | — | — | — | — |
| Example A | | | | | | |
| Perfume from | — | 0.51 | — | — | — | — |
| Example C | | | | | | |
| Perfume from | — | — | 0.51 | — | — | 0.51 |
| Example D | | | | | | |
| Perfume from | — | — | — | 0.51 | — | — |
| Example H | | | | | | |
| Perfume from | — | — | — | — | 0.51 | — |
| example E | | | | | | |
| pH @ 10% | | 9 | 9 | 9 | 9 | 9 |

¹C12-13 alkyl ethoxy sulfonate containing an average of 0.6 ethoxy groups.
²C₁₂-C₁₄ Amine oxide.
³Nonionic may be either C11 Alkyl ethoxylated surfactant containing 9 ethoxy groups or C10 Alkyl ethoxylated surfactant containing 8 ethoxy groups.
⁴Polymer is (N,N-dimethylamino)ethyl methacrylate homopolymer
⁵1,3 bis(methylamine)-cyclohexane.
⁶The protease is selected from: Savinase ®; Maxatase ®; Maxacal ®; Maxapem 15 ®; subtilisin BPN and BPN′; Protease B; Protease A; Protease D; Primase ®; Durazym ®; Opticlean ®; and Optimase ®; and Alcalase ®.

Examples 7-10 describes Light Duty Liquid dishwashing detergents according to the present invention:

| | Example | Example | Example | Example |
|---|---|---|---|---|
| | 7 | 8 | 9 | 10 |
| AE0.6S¹ | 26.09 | 26.09 | 26.09 | 28.80 |
| Amine oxide² | 6.50 | 6.5 | 8.0 | 8.0 |
| Suds boosting | 0.20 | 0.20 | 0.20 | 0.22 |
| polymer³ | | | | |
| Sodium Cumene | 3.50 | 3.50 | 3.50 | 3.90 |
| Sulfonate | | | | |
| Nonionic⁴ | 3.00 | 3.00 | 3.00 | 3.30 |
| Diamine⁵ | 0.50 | 0.50 | 0.50 | 0.55 |
| Sodium Chloride | 1.5 | 1.5 | 1.5 | 1.5 |
| NaOH | 0.35 | 0.35 | 0.35 | 0.35 |
| Na₂CO₃ | 1.75 | 1.75 | 1.75 | 1.75 |
| K₂CO₃ | 0.75 | 0.75 | 0.75 | 0.75 |
| propylene glycol | 4.0 | 4.0 | 4.0 | 4.0 |
| polypropylene glycol | 1.0 | 1.0 | 1.0 | 1.0 |
| Ethanol | 3.0 | 0.7 | 0.7 | — |
| Perfume from | 0.51 | — | — | — |
| example B | | | | |
| Perfume from | — | 0.51 | — | — |
| Example A | | | | |
| Perfume from | — | — | 0.51 | — |
| example C | | | | |
| Perfume from | — | — | — | 0.51 |
| Example E | | | | |
| Water and Misc. | BAL. | BAL. | BAL. | BAL. |
| Viscosity (cps @ 70 F.) | 353 | 640 | 635 | 848 |
| pH @ 10% | 10.8 | 10.8 | 10.80 | 10.8 |

¹C12-13 alkyl ethoxy sulfonate containing an average of 0.6 ethoxy groups.
²C₁₂-C₁₄ Amine oxide.
³Polymer is (N,N-dimethylamino)ethyl methacrylate homopolymer
⁴Nonionic may be either C10 Alkyl ethoxylated surfactant containing 8 ethoxy groups or C10 Alkyl ethoxylated surfactant containing 8 ethoxy groups.
⁵1,3 bis(methylamine)-cyclohexane

What is claimed is:

1. A perfume composition comprising:
a) at least 7.5% by weight of the composition of a first perfume ingredient having boiling point of 250° C. or less and ClogP of 3.0 or less, and;
b) at least 35% by weight of the composition of a second perfume ingredient having boiling point of 250° C. or less and ClogP of 3.0 or more,
the composition being further characterized in that at least one, first or second perfume ingredient is present in an amount of at least 7% by weight of the composition.

2. A perfume composition according to claim 1 wherein the first perfume ingredient has ClogP of less than 3.0.

3. A perfume composition according to claim 1 wherein the second perfume ingredient has a Clog P of greater than 3.0.

4. A perfume composition according to claim 1 comprising from at least 8.5% to at least 9.5% by weight of the composition of a first perfume ingredient.

5. A perfume composition according to claim 1 comprising from at least 37.5% to at least 40% by weight of the composition of a second perfume ingredient.

6. A perfume composition according to claim 1 wherein at least one, first and/or second perfume ingredient is present at a level of from at least 8.5% to at least 10% by weight of the composition.

7. A perfume composition according to claim 1 wherein the first and/or second perfume ingredient is selected from the group consisting of esters, ketones, aldehydes, alcohols.

8. A perfume composition according to claim 1 wherein the at least one, first and/or second perfume ingredient is present at a level exceeding the Odor Detection Range (ODR) of the ingredient.

9. A perfume composition according to claim 1 wherein at least one, first and/or second perfume ingredient is present at a level at least 50% higher than the ODR.

10. A perfume composition according to claim 1 wherein at least one, first and/or second perfume ingredient is present at a level at least 150% higher than the ODR.

11. A perfume composition according to claim 1 wherein at least one, first and/or second perfume ingredient is present at a level at least 300% higher than the ODR.

12. A cleaning composition comprising the perfume composition according to claim 1.

13. A cleaning composition according to claim 13 additionally comprising ethanol, preferably at a level of greater than 3% by weight of the composition.

14. A method of using a cleaning composition according to claim 12 or 13 for cleaning dishware, cutlery and cooking utensils.
